(19) United States
(12) Patent Application Publication
(10) Pub. No.: US 2022/0346833 A1
(43) Pub. Date: Nov. 3, 2022

(54) SURGICAL ACCESS DEVICE WITH TEXTURED BALLOON

(71) Applicant: Covidien LP, Mansfield, MA (US)

(72) Inventors: Jason Mickus, Avon, CT (US); Kevin M. Desjardin, Prospect, CT (US); Astley C. Lobo, West Haven, CT (US)

(21) Appl. No.: 17/246,948

(22) Filed: May 3, 2021

(51) Int. Cl. A61B 17/34

(52) U.S. Cl. CPC A61B 17/3423; A61B 2017/3486

(57) ABSTRACT

A surgical access device is insertable into tissue and includes a housing and a tubular member extending from the housing. A balloon is attached to an outer surface of the tubular member. The balloon has an outer surface with a plurality of depressions thereon. The plurality of depressions is arranged such that a portion of the balloon has a contact patch with an area that is less than an area of tissue surrounding the contact patch. The contact patch of the balloon has surface area that is greater than the area of tissue surrounding the contact patch of the balloon.

### FIELD

The present disclosure generally relates to surgical access devices. In particular, the present disclosure relates to a surgical access device with a textured balloon.

### BACKGROUND

In minimally invasive surgical procedures, including endoscopic and laparoscopic surgeries, a surgical access device permits the introduction of a variety of surgical instruments into a body cavity or opening. A surgical access device (e.g., a cannula or an access port) is introduced through an opening in tissue (e.g., a naturally occurring orifice or an incision) to provide access to an underlying surgical site in the body. The opening is typically made using an obturator having a blunt or sharp tip that may be inserted through a passageway of the surgical access device. For example, a cannula has a tube of rigid material with a thin wall construction, through which an obturator may be passed. The obturator is utilized to penetrate a body wall, such as an abdominal wall, or to introduce the surgical access device through the body wall, and is then removed to permit introduction of surgical instruments through the surgical access device to perform the surgical procedure.

Minimally invasive surgical procedures, including both endoscopic and laparoscopic procedures, permit surgery to be performed on organs, tissues, and vessels far removed from an opening within the tissue. In laparoscopic procedures, the abdominal cavity is insufflated with an insufflation gas, e.g., CO₂, to create a pneumoperitoneum thereby providing access to the underlying organs. A laparoscopic instrument is introduced through a cannula into the abdominal cavity to perform one or more surgical tasks. The cannula may incorporate a seal to establish a substantially fluid tight seal about the laparoscopic instrument to preserve the integrity of the pneumoperitoneum. The cannula, which is subjected to the pressurized environment, e.g., the pneumoperitoneum, may include an inflatable anchor to prevent the cannula from backing out of the opening in the abdominal wall, for example, during withdrawal of the laparoscopic instrument from the cannula. Typically, the surgical access device includes separate valves for insufflating the abdominal cavity and for inflating/deflating the inflatable anchor.

### SUMMARY

A surgical access device according to the present disclosure is configured for insertion into tissue and includes a housing and a tubular member extending from the housing. A balloon is attached to an outer surface of the tubular member and has an outer surface with a plurality of depressions thereon. The plurality of depressions is arranged such that a portion of the balloon has a contact patch with an area that is less than an area of tissue surrounding the contact patch and a surface area that is greater than the area of tissue surrounding the contact patch.

In an aspect of the present disclosure, the difference between the area of the contact patch and the area of tissue surrounding the contact patch may reduce an insertion force of the surgical access device.

In aspects of the present disclosure, each depression of the plurality of depressions may be a dimple.

In another aspect of the present disclosure, each depression of the plurality of depressions may have a hexagonal configuration.

In a further aspect of the present disclosure, each depression of the plurality of depressions may have an irregular configuration.

In yet another aspect of the present disclosure, the surgical access device may further include an inflation assembly coupling a source of inflation fluid with the balloon.

In an aspect of the present disclosure, a contact area of an expandable portion of the balloon may be greater than a contact area of tissue adjacent the expandable portion when inflation fluid is introduced into the balloon thereby minimizing movement of the surgical access device relative to surrounding tissue.

In aspects of the present disclosure, the contact area of the expandable portion may be equal to the surface area of the expandable portion.

A surgical access device according to an aspect of the present disclosure is configured for insertion through an opening in tissue and includes a housing with a tubular member extending from the housing. A balloon is attached to an outer surface of the tubular member. The balloon includes a proximal portion, a distal portion, and an expandable portion disposed therebetween. The expandable portion is transitionable between a collapsed configuration and an expanded configuration and the balloon has an outer surface with a plurality of depressions disposed between the proximal portion and the distal portion. The plurality of depressions is arranged such that a portion of the balloon has a contact patch with an area that is less than an area of tissue surrounding the contact patch and a surface area that is greater than the area of tissue surrounding the contact patch.

In an aspect of the present disclosure, the difference between the area of the contact patch and the area of tissue surrounding the contact patch may reduce an insertion force of the surgical access device.

In one aspect of the present disclosure, each depression of the plurality of depressions may be a dimple.

In another aspect of the present disclosure, each depression of the plurality of depressions may have a hexagonal configuration.

In a further aspect of the present disclosure, each depression of the plurality of depressions may have an irregular configuration.

In aspects of the present disclosure, the surgical access device may further include an inflation assembly fluidly coupling an inflation source with the expandable portion of the balloon.

In yet another aspect of the present disclosure, the expanded configuration of the expandable portion may have a contact area equal to its surface area and greater than a contact area of tissue adjacent the expandable portion.

Other features of the disclosure will be appreciated from the following description.

### DESCRIPTION OF THE DRAWINGS

The accompanying drawings, which are incorporated in and constitute a part of this specification, illustrate aspects and features of the disclosure and, together with the detailed description below, serve to further explain the disclosure, in which:

FIG. 1 is a perspective view of a surgical access device including a balloon according to the present disclosure;

FIG. 1A is a side cross-sectional view of the surgical access device of FIG. 1 taken along section line 1A-1A with an expandable portion of the balloon in an expanded configuration;

FIG. 2 is a perspective view of a balloon of the surgical access device of FIG. 1;

FIG. 3 is an enlarged view of the area of detail of FIG. 2 showing surface features of the balloon;

FIG. 4 is an alternate configuration of the surface features shown in FIG. 3;

FIG. 5 is another alternate configuration of the surface features shown in FIG. 3; and

FIG. 6 is a side view of the surgical access device of FIG. 1 inserted through tissue with a collar of the surgical access device abutting tissue and an expandable portion of the balloon in an expanded configuration.

### DETAILED DESCRIPTION

Aspects of the disclosure are described hereinbelow with reference to the accompanying drawings; however, it is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

Descriptions of technical features of an aspect of the disclosure should typically be considered as available and applicable to other similar features of another aspect of the disclosure. Accordingly, technical features described herein according to one aspect of the disclosure may be applicable to other aspects of the disclosure, and thus duplicative descriptions may be omitted herein. Like reference numerals may refer to like elements throughout the specification and drawings.

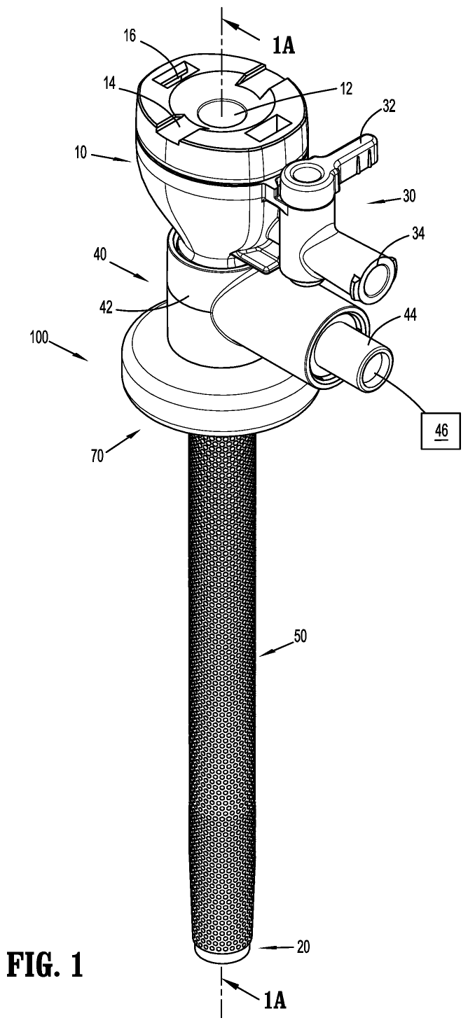

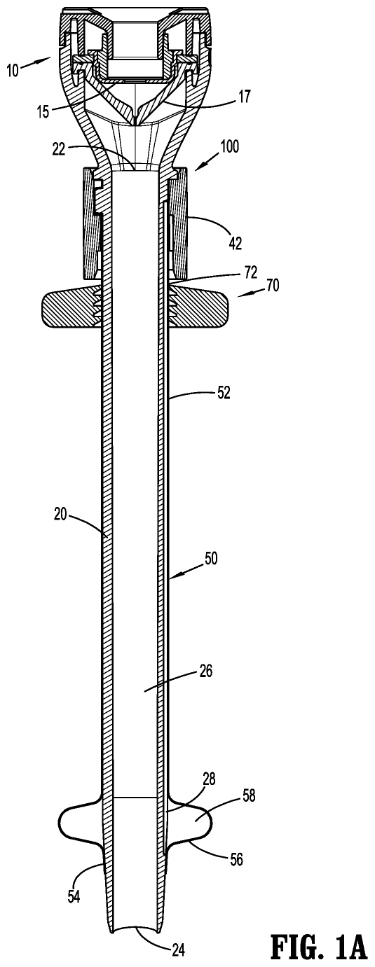

With initial reference to FIGS. 1 and 1A, a surgical access device according to the present disclosure is illustrated as surgical access device 100. The surgical access device 100 includes a housing 10 and a tubular member 20 extending distally from the housing 10. A balloon 50 is attached to an outer surface of the tubular member 20 and extends along a majority of a length of the tubular member 20. The housing 10 has a proximal opening 12 that is configured to receive a surgical instrument (not shown) therethrough. The housing 10 also has opposed notches 14 and recesses 16 that are adapted for releasably engaging an obturator or trocar (not shown). An insufflation valve assembly 30 extends radially from the housing 10 and includes a rotatable valve 32 and a port 34 with a luer connection. The valve 32 is rotatable between an open position and a closed position. The valve 32 may be a stopcock valve. Further, the housing 10 includes an instrument seal 15 that is adapted to engage a surgical instrument (not shown) inserted through the housing 10 and provide a fluid-tight seal therewith. Additionally, the housing 10 includes a zero-closure seal 17 (e.g., a duckbill seal) that allows fluid flow through the housing 10 towards a distal opening 24 of the tubular member 20 and inhibits proximal fluid flow in the absence of a surgical instrument. The tubular member 20 is attached to the housing 10 using an adhesive or by ultrasonic welding.

The tubular member 20 has opposed proximal and distal openings 22, 24 defining a lumen 26 therethrough. The lumen 26 is in fluid communication with the proximal opening 12 of the housing 10 and the insufflation valve assembly 30. An inflation assembly 40 is disposed distally of the housing 10 and circumscribes the tubular member 20. An inflation port 44 extends radially from a ring 42 of the inflation assembly 40 and is configured to receive an inflation fluid (e.g., air, CO₂, etc.) from an inflation source 46 (e.g., syringe, pump, etc.) and provide a path for the inflation fluid to enter a chamber 58 of the balloon 50. The balloon 50 has a proximal sleeve or proximal portion 52, a distal sleeve or distal portion 54, and an expandable portion 56 disposed between the proximal and distal portions 52, 54. The expandable portion 56 is transitionable between a collapsed configuration (FIG. 1) and an expanded configuration (FIG. 1A). The balloon 50 is attached to the tubular member 20 using an adhesive or by ultrasonic welding. The chamber 58 of the balloon 50 is defined between an inner surface of the expandable portion 56 of the balloon and an outer surface of the tubular member 20. Inflation fluid flows through the inflation port 44 and a channel 28 of the tubular member 20 and into the chamber 58 of the balloon 50. The channel 28 of the tubular member 20 is a recess formed in the outer surface of the tubular member 20 that extends between the ring 42 of the inflation assembly 40 and the chamber 58 of balloon 50. A collar 70 is slidably disposed on the tubular member 20. The collar 70 has a generally circular configuration with a central opening 72 and circumferentially surrounds an outer surface of the balloon 50 and therefore the outer surface of the tubular member 20. The collar 70 is repositionable along the tubular member 20 between a proximal position near the inflation assembly 40 and a distal position near the expandable portion 56 of the balloon 50. The collar 70 aids maintaining a position of the surgical access device 100 in tissue as will be discussed in detail hereinafter. The collar 70 may be formed from a compressible material (e.g., foam, cotton or other suitable textile) or an elastomeric material (e.g., rubber or silicone) to aid in sealing the opening into the tissue of the body wall and anchoring the surgical access device 100 in a patient. The collar 70 may be any known retention mechanism used on cannulas and/or trocars, such as foam or rubber collars. Examples of suitable surgical access devices are disclosed in commonly owned U.S. Pat. Nos. 7,300,448; 7,691,089; and 10,022,149, the entire disclosures of which are hereby incorporated by reference.

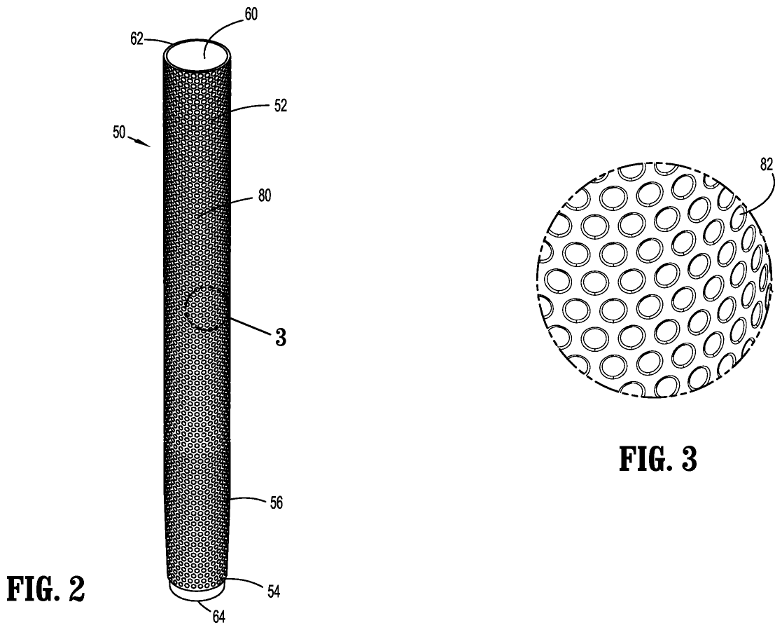

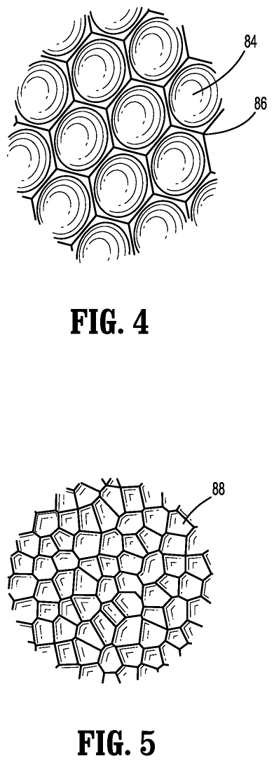

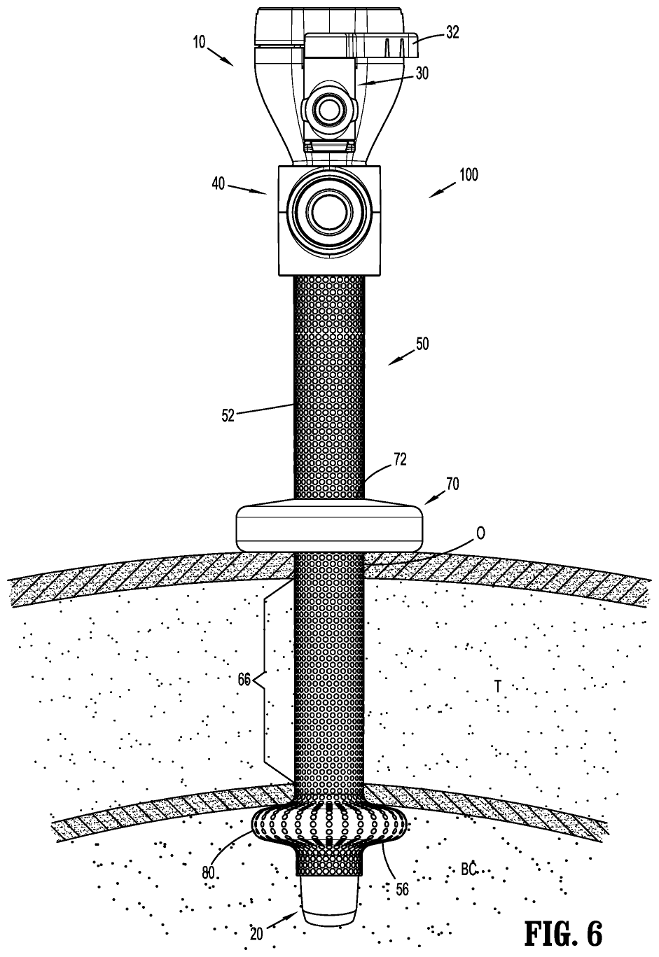

With reference now to FIGS. 2-5, additional features of the balloon 50 are illustrated. The balloon 50 has a generally elongated and cylindrical configuration. A passage 60 extends between open proximal and distal ends 62, 64 of the balloon 50. The passage 60 has an inner diameter that is slightly greater than an outer diameter of the tubular member 20 such that there is a snug fit when the balloon 50 is attached to the tubular member 20. This facilitates maintaining the position of the balloon 50 relative to the tubular member 20 prior to bonding the balloon 50 to the tubular member 20. The area of detail identified in FIG. 2 illustrates surface features of the balloon. The surface features are depressions 80 in the material of the balloon 50 and are distributed along the outer surface of the balloon 50. As shown, the depressions 80 are uniformly distributed on the outer surface of the balloon 50. It is contemplated that the depressions 80 may be distributed in a non-uniform arrangement. The depressions 80 may be dimples 82 as shown in FIG. 3. In this configuration, each dimple 82 is a generally hemispherical pocket in the balloon 50 with spacing between each dimple 82. In an alternate configuration, as seen in FIG. 4, the depressions 80 are concavities 84 in the balloon 50 having a generally hemispherical configuration and each concavity 84 is bounded by a hexagon 86 resulting in a pattern resembling a honeycomb. Another configuration, shown in FIG. 5, is a more random pattern of recesses 88 that are adjacent to one another and distributed in an irregular pattern. These recesses 88 do not have a uniform configuration in contrast to the arrangement of the dimples 82 of FIG. 3 or the hexagonal configuration of the concavities 84 of FIG. 4. In each instance, the depressions 80 reduce the area of a contact patch 66 of the balloon 50 that engages a section of tissue “T” (FIG. 6) surrounding the contact patch 66 while simultaneously increasing the surface area of the balloon 50. The area of the contact patch 66 is less than the area of tissue “T” surrounding the contact patch 66. Since there is no contact between tissue “T” and the depressions 80 of the balloon 50 due to their recessed nature, while there is contact between tissue “T” and areas of the balloon 50 that surround the depressions 80 (i.e., the contact patch 66), the reduced surface interaction between the balloon 50 and tissue “T” surrounding the balloon 50 facilitates reduced insertion and removal forces. Reducing the contact patch 66 reduces the frictional forces between the balloon 50 and the tissue “T” surrounding the balloon 50, which facilitates insertion and removal of the surgical access device 100 by reducing the amount of force needed to move the tubular member 20 and the balloon 50 through tissue “T”. The surface area of the balloon 50 having depressions 80 is greater than the surface area of a balloon without depressions when both balloons have the same length and diameter. Increasing the surface area of the balloon 50 and particularly the surface area of the expandable portion 56 of the balloon 50 aids in retaining the surgical access device 100 as will be discussed hereinbelow.

Referring now to FIG. 6, the surgical access device 100 is shown positioned and anchored in a patient. The tubular member 20 of the surgical access device 100 is inserted through an opening “O” in the patient's tissue “T”. The tubular member 20 extends through tissue “T” and provides access to a surgical site in the body cavity “BC”. Once the tubular member 20 of the surgical access device 100 is positioned in a desired location, inflation fluid from the inflation source 46 (FIG. 1) is supplied to the chamber 58 of the expandable portion 56 of the balloon 50 thereby transitioning the expandable portion 50 from the collapsed configuration (FIG. 1) to the expanded configuration (FIG. 6). The collar 70 is slid distally along the outer surface of the balloon 50 until it contacts an outer surface of tissue “T” thereby sandwiching tissue “T” between the collar 70 and the expandable portion 56 of the balloon 50. In the expanded configuration, the expandable portion 56 of the balloon 50 abuts tissue “T” adjacent the body cavity “BC” and, in cooperation with the collar 70, anchors the surgical access device 100. Further, the expanded configuration of the expandable portion 56 everts the depressions 80 in the expandable portion 56 thereby increasing the contact area of the expandable portion 56 to equal the surface area of the expandable portion 56 which is greater than a contact area of tissue adjacent to the expandable portion 56. This increased contact between the expandable portion 56 of the balloon 50 in the expanded configuration and adjacent tissue improves stability of the surgical access device 100 and increases the resistance of the surgical access device 100 to inadvertent movement during a surgical procedure.

Persons skilled in the art will understand that the devices and methods specifically described herein and illustrated in the accompanying drawings are non-limiting. It is envisioned that the elements and features may be combined with the elements and features of another without departing from the scope of the disclosure. As well, one skilled in the art will appreciate further features and advantages of the disclosure.

What is clamed is:

1. A surgical access device configured for insertion into tissue, the surgical access device comprising:
a housing;
a tubular member extending from the housing; and
a balloon attached to an outer surface of the tubular member, the balloon having an outer surface with a plurality of depressions thereon, the plurality of depressions arranged such that a portion of the balloon has a contact patch, the contact patch having an area that is less than an area of tissue surrounding the contact patch and a surface area that is greater than the area of tissue surrounding the contact patch.

2. The surgical access device according to claim 1, wherein the difference between the area of the contact patch and the area of tissue surrounding the contact patch reduces an insertion force of the surgical access device.

3. The surgical access device according to claim 2, wherein each depression of the plurality of depressions is a dimple.

4. The surgical access device according to claim 2, wherein each depression of the plurality of depressions has a hexagonal configuration.

5. The surgical access device according to claim 2, wherein depression of the plurality of depressions has an irregular configuration.

6. The surgical access device according to claim 1, further including an inflation assembly coupling a source of inflation fluid with the balloon.

7. The surgical access device according to claim 6, wherein a contact area of an expandable portion of the balloon is greater than a contact area of tissue adjacent the expandable portion when inflation fluid is introduced into the balloon thereby minimizing movement of the surgical access device relative to surrounding tissue.

8. The surgical access device according to claim 7, wherein the contact area of the expandable portion is equal to the surface area of the expandable portion.

9. A surgical access device configured for insertion through an opening in tissue, the surgical access device comprising:
a housing;
a tubular member extending from the housing; and
a balloon attached to an outer surface of the tubular member, the balloon including a proximal portion, a distal portion, and an expandable portion disposed therebetween, the expandable portion transitionable between a collapsed configuration and an expanded configuration, the balloon having an outer surface with a plurality of depressions disposed between the proximal portion and the distal portion, the plurality of depressions arranged such that a portion of the balloon has a contact patch with an area that is less than an area of tissue surrounding the contact patch and a surface area that is greater than the area of tissue surrounding the contact patch.

10. The surgical access device according to claim 9, wherein the difference between the area of the contact patch and the area of tissue surrounding the contact patch reduces an insertion force of the surgical access device.

11. The surgical access device according to claim 10, wherein each depression of the plurality of depressions is a dimple.

12. The surgical access device according to claim 10, wherein each depression of the plurality of depressions has a hexagonal configuration.

13. The surgical access device according to claim 10, wherein each depression of the plurality of depressions has an irregular configuration.

14. The surgical access device according to claim 9, further including an inflation assembly fluidly coupling an inflation source with the expandable portion of the balloon.

15. The surgical access device according to claim 14, wherein the expanded configuration of the expandable portion has a contact area equal to its surface area and greater than a contact area of tissue adjacent the expandable portion.
